(19) United States
(12) Patent Application Publication
(10) Pub. No.: US 2005/0239688 A1
(43) Pub. Date: Oct. 27, 2005

(54) Method for the synthesis of anthracycline-peptide conjugates

(76) Inventors: Anne-Marie Fernandez, Bruxelles (BE); Vincent Dubois, Gif sur Yvette (FR)

Correspondence Address: KNOBBE MARTENS OLSON & BEAR LLP, 2040 MAIN STREET, FOURTEENTH FLOOR, IRVINE, CA, 92614, US

(21) Appl. No.: 10/522,565

(22) PCT Filed: Jul. 23, 2003
(86) PCT No.: PCT/EP03/08082

§ 371 Date: Jun. 20, 2005

(30) Foreign Application Priority Data
Jul. 24, 2002 (EP) 02447145.0

(51) Int. Cl. A61K 38/14; C07K 9/00; A61K 31/704; C07H 15/24

(52) U.S. Cl. 514/8; 514/34; 530/322; 536/6.4

(57) ABSTRACT

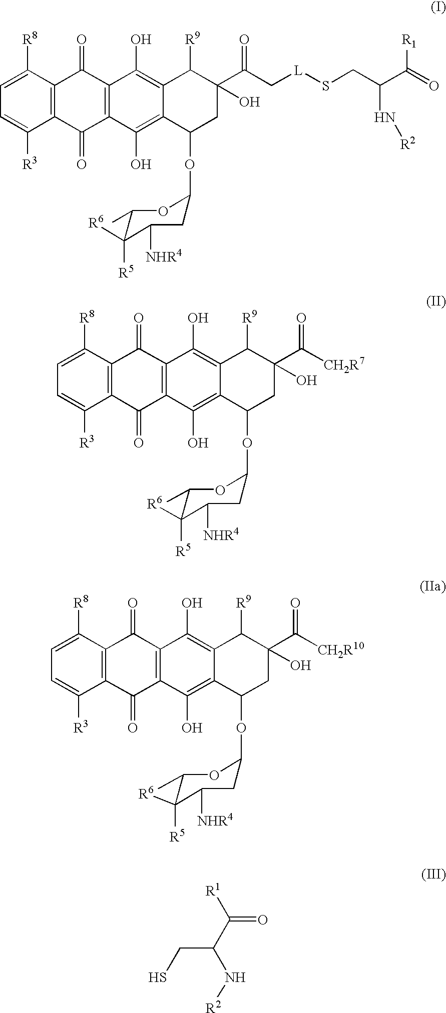

The present invention relates to a method for the preparation of a compound of formula (I) or pharmaceutically acceptable salts thereof and intermediates thereof, comprising the steps of: a) halogenating a compound of formula (II), resulting in compound of formula (IIa), b) reacting a compound of formula (IIa) at its 14 position with the thiol moiety of a peptide of formula (III), optionally in the presence of a suitable linker, to obtain said compound of formula (I), wherein R₁ represents OH, NH₂ or NH-peptide; R² represents H or —CO-peptide; R³ represents OCH₃, OH or H; R⁴ represents H, or COCF₃; R⁵ represents OH, O-tetrahydropyranyl or H; R⁶ represents OH or H; R⁷ represents H, OH, OCO(CH₂)₃CH₃ or OCOCH(OC₂H₅)₂; R⁸ represents OH or H; R⁹ represents OH or H; R¹⁰ represents a halogen and L is an optional suitable linker arm.

### FIELD OF THE INVENTION

The present invention relates to a method for the synthesis of anthracycline-peptide conjugates. More in particular the present invention relates to a method for the synthesis of doxorubicin-peptide conjugates. The present invention further relates to anthracycline-peptide conjugates or pharmaceutically acceptable salt thereof obtained by said methods. Said invention further relates to the use of said anthracycline-peptide conjugates as medicaments for treating cancer.

### BACKGROUND OF THE INVENTION

Anthracycline compounds are among the most effective and widely used antitumor agents. The best-known members of this class of compounds are doxorubicin and daunorubicin. Daunorubicin is effective in treating acute leukemia. Doxorubicin is one of the most active antineoplastic ever identified. It is known to treat acute leukemia, Hodgkin's disease and non-Hodgkin's lymphomas, small cell and non-small cell lung cancer, cancers of the breast, ovaries, stomach, thyroid, and bladder, osteogenic and soft tissue sarcomas, and malignant melanoma. Although these compounds may be useful in the treatment of neoplasms and other disease states wherein a selected cell population is sought to be eliminated, their therapeutic efficacy is often limited by the dose-dependent toxicity associated with their administration. Furthermore, the existence of drug resistance in tumors results in decreased cytotoxicity of these compounds.

Peptide conjugates of anthracyclines are known, and different methods for their synthesis have been described. WO 00/78359 relates to a method and composition for treating cancer and chemotherapy-resistant cancers comprising an anthracycline conjugated to or co-administrated with a peptide. Therein the peptide is linked to the anthracycline either through an amide bond between the amino terminus of doxorubicin and the carboxy terminus of said peptide, or through an ester bond between the primary hydroxyl of doxorubicin and the carboxy terminus of said peptide. U.S. Pat. No. 5,998,362 relates to chemical conjugates which comprises oligopeptides and know cytotoxic agents such as anthracyclines. Said oligopeptides are covalently attached either at the amino terminus or at the 14-hydroxyl of the anthracycline. Although several useful new derivatives have been synthesized, there is still an urgent need to find analogues that can be easily prepared and in high quantities.

It is an object of the invention to provide new methods for the synthesis of anthracycline-peptide conjugates. It is another object of the present invention to provide easy to implement methods for the synthesis of said conjugates. It a further object to provide methods for the synthesis of said conjugates comprising a limited number of steps. It is yet another object to provide methods wherein said conjugates can be prepared cheaply from readily available starting materials and reagents. It is a further object to provide method for the synthesis of said conjugates wherein said conjugates are produced with good yields. It is another object of the invention to provide new anthracycline-peptide conjugates which are potent antitumor agents. It is yet another object of the invention to provide new anthracycline-peptide conjugates, which are useful in the treatment of multidrug resistant tumor.

### SUMMARY OF THE INVENTION

According to a first aspect, the present invention relates to methods for the synthesis of anthracycline-peptide conjugates of formula (I) or pharmaceutically acceptable salts thereof and intermediates thereof, 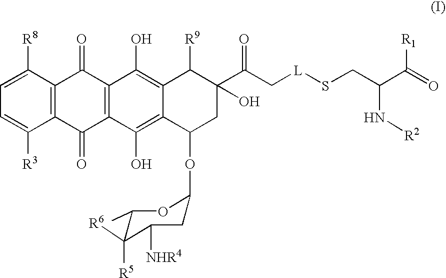
wherein said method comprises the steps of reacting a compound of formula (II) at its 14 position with the thiol moiety of a peptide of formula (III), optionally in the presence of a suitable linker, to obtain said compound of formula (I) wherein R³ represents OCH₃, OH or H; R⁴ represents H, or COCF₃; R⁵ represents OH, O-tetrahydropyranyl or H; R⁶ represents OH or H; R⁷ represents H, OH, OCO(CH₂)₃CH₃ or OCOCH(OC₂H₅)₂; R⁸ represents OH or H; R⁹ represents OH or H; R¹ represents OH, NH₂ or NH-peptide; R² represents H or —CO-peptide; and L is a suitable optional linker arm.

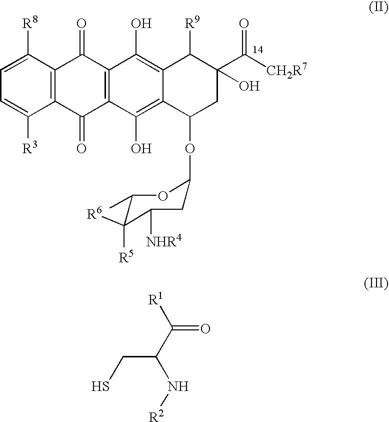

More in particular, the present invention relates to methods for the preparation of a compound of formula (I) or pharmaceutically acceptable salts thereof and intermediates thereof, comprising the steps of first halogenating a compound of formula (II), resulting in compound of formula (IIa), 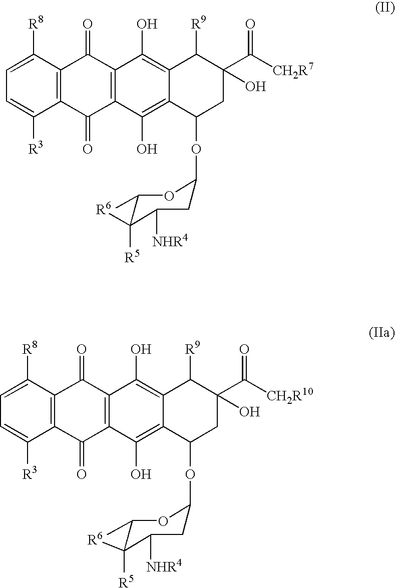
secondly reacting a compound of formula (lla) at its 14 position with the thiol moiety of a peptide of formula (III), optionally in the presence of a suitable linker, to obtain said compound of formula (I) 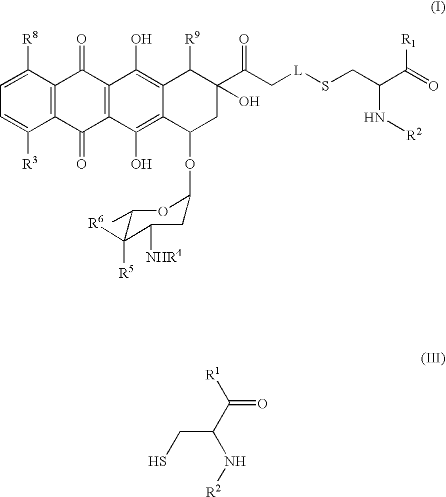
wherein R¹ represents OH, NH₂ or NH-peptide; R² represents H or —CO-peptide; R³ represents OCH₃, OH or H; R⁴ represents H, or COCF₃; R⁵ represents OH, O-tetrahydropyranyl or H; R⁶ represents OH or H; R⁷ represents H, OH, OCO(CH₂)₃CH₃ or OCOCH(OC₂H₅)₂; R⁸ represents OH or H; R⁹ represents OH or H; R¹⁰ represents a halogen and L is a suitable optional linker arm.

According to an embodiment the present invention relates to a method wherein, said compound of formula (IIa) is reacted at its 14 position with a linker of formula (IV) to obtain compound of formula (V), wherein Z is a functional group able to react with a thiol, and X is a bivalent radical selected from the group comprising an alkyl, an aralkyl, an alkenyl, a cycloalkyl and an aryl radical; 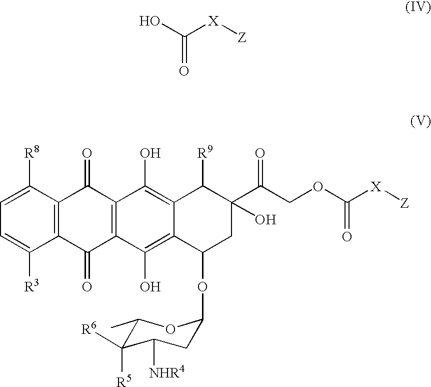
the compound of formula (V) is then coupled with the thiol moiety of a peptide of formula (III) to obtain the compound of formula (I), 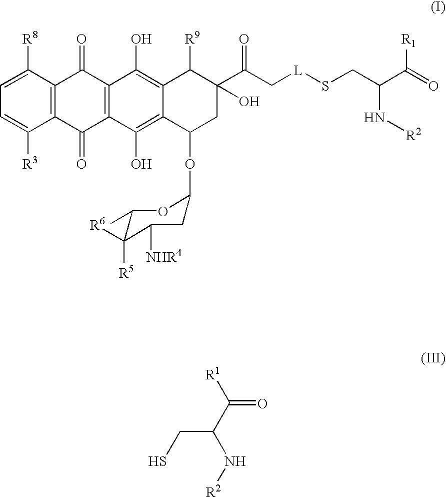
wherein L represents a linker arm of the formula R—X—Y—, wherein R is —O—C(═O)—, Y is the product of Z upon reaction with the thiol moiety of compound of formula (III) and X, R¹, R², R³, R⁴, R⁵, R⁶, R⁸ and R⁹ have the same meaning as that defined above.

According to another embodiment, the present invention relates to a method wherein said compound of formula (IIa) is directly reacted at its 14 position with the thiol moiety of a peptide of formula (III) to obtain compound of formula (I) wherein R¹, R², R³, R⁴, R⁵, R⁶, R⁸, R⁹ have the same meaning as that defined above and L is absent as represented by compound of formula (Ia).

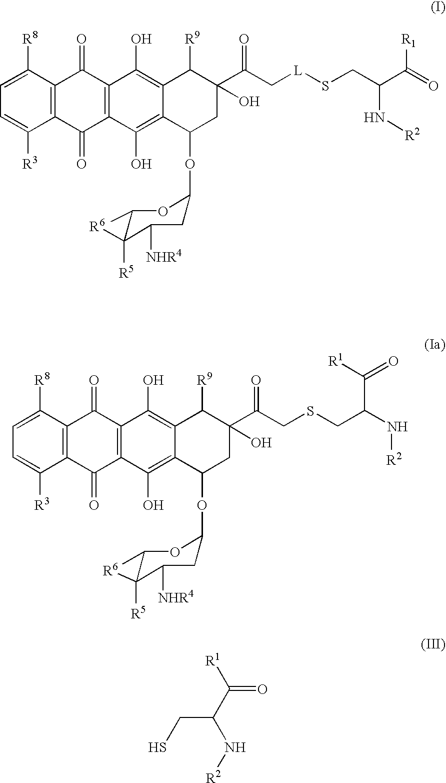

The present invention further relates in a second aspect to anthracycline-peptide conjugates and intermediates obtained by said methods. Said anthracycline-peptide conjugate of formula (I) comprises a peptide containing at least one cysteine which is covalently linked to the 14-carbon group of said anthracycline via the side chain of said cysteine residue, optionally through a suitable linker.

Furthermore, the present invention relates to the use of said new anthracycline-peptide conjugates as medicaments in the treatment of cancer.

The present invention will be further disclosed in detail hereunder. Examples are given which will further support the description.

### DETAILED DESCRIPTION

The present invention relates to methods for the synthesis of anthracycline-peptide conjugate of formula (I) or pharmaceutically acceptable salt thereof, wherein the peptide is covalently linked to the 14-carbon group of said anthracycline via the side chain of a cysteine residue, optionally through a suitable bifunctional linker L.

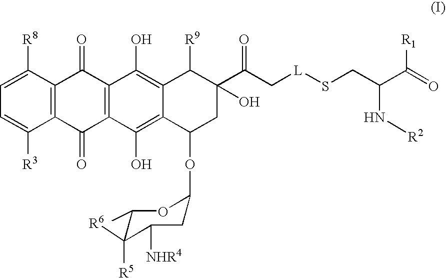

The linker arm L in compound of formula (I) may represents any bivalent radical between the methyl group (C14) and the thioether group in compound of formula (I). L is preferably of the formula R—X—Y—, wherein R represents an ester bond, X represents a bivalent radical selected from the group comprising an alkyl, an aralkyl, an alkenyl, a cycloalkyl and an aryl radical and Y is a functional group selected from the group comprising carbonyl, carboxy, carbamoyl and imidyl radical, or L may be absent in compound of formula (I) as illustrated by formula (Ia).

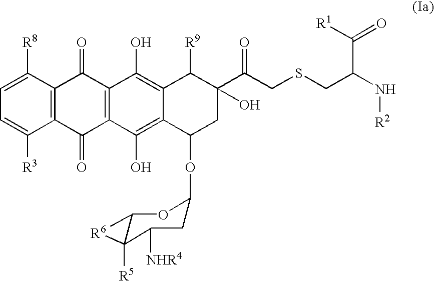

As used herein the term “alkyl” and the alkyl portion of aralkyl and similar terms, refers to saturated bivalent hydrocarbon radicals having straight, branched or cyclic moieties or combinations thereof and contains 1-20 carbon atoms, preferably 1-10 carbon atoms, more preferably 1-8 carbon atoms, still more preferably 1-6 carbon atoms, yet more preferably 1-4 carbon atoms. Preferred alkyl radicals are methyl, ethyl, propyl, isopropyl, n-butyl, isobutyl, pentyl, isoamyl, hexyl, cyclohexyl and the like. The term “aryl” as used herein, includes a bivalent organic radical derived from an aromatic hydrocarbon by removal of two hydrogen, and includes any monocyclic or bicyclic carbon ring of up to 7 members in each ring, wherein at least one ring is aromatic. Examples of such aryl elements include phenyl, naphthyl, tetrahydronaphthyl, indanyl, biphenyl, phenanthryl, anthryl or acenaphthyl. The term “aralkyl” as used herein, relates to a group of the formula alkyl-aryl in which alkyl is as defined above. Examples of aralkyl radicals include benzyl, phenethyl and the like. The term “cycloalkyl” as used herein is intended to include bivalent non-aromatic cyclic hydrocarbon groups. Examples of cycloalkyl groups include cyclopropyl, cyclobutyl, cyclopentyl, cyclohexyl and the like. The term “alkenyl” as used herein, includes bivalent hydrocarbon radicals having one or several double bonds, having straight, branched or cyclic moieties or combinations thereof and contains 2-20 carbon atoms, preferably 2-10 carbon atoms, more preferably 2-8 carbon atoms, still more preferably 2-6 carbon atoms, yet more preferably 24 carbon atoms Examples of alkenyl groups include vinyl, allyl, isopropenyl, pentenyl, hexenyl, heptenyl, cyclopropenyl, cyclobutenyl, cyclopentenyl, cyclohexenyl, 1-propenyl, 2-butenyl, 2-methyl-2-butenyl, isoprenyl, farnesyl, geranyl, geranylgeranyl and the like.

The term “carbonyl” as used herein refers to a bivalent radical of formula —C(═O)alkyl-, being a straight, branched or cyclic radical or combinations thereof. The term “carboxy” as used herein refers to a bivalent radical of formula —C(═O)O-alkyl being a straight, branched or cyclic radical or combinations thereof. The term “carbamoyl” as used herein refers to a bivalent radical of formula —N(alkyl)C(═O)O-alkyl- being a straight, branched or cyclic radical or combinations thereof. The term “imidyl” as used herein refers to a bivalent radical of formula —N(C(═O)-alkyl)₂-being a straight, branched or cyclic radical or combinations thereof such as succinimide.

As used herein “compound”, includes within its scope not just the specific compound(s) listed or described but also alternative forms of the compound. The compounds may have asymmetric centers, occur as racemates, racemic mixtures, and as individual diastereoisomers, with all possible stereochemical isomers including optical isomers, being included in the present invention.

The starting material in said methods is an anthracycline, more preferably an anthracycline of formula (II), wherein R⁷ represents H, OH, —OCO(CH₂)₃CH₃ or —OCOCH(OC₂H₅)₂, and R³, R⁴, R⁵, R⁶, R⁸ and R⁹ have the same meaning as that defined above.

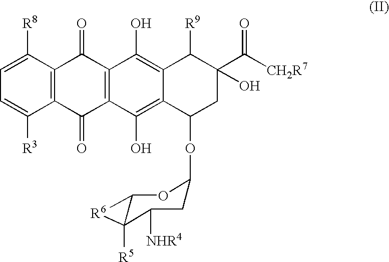

According to a preferred embodiment, said anthracycline of formula (II) is selected from the group comprising doxorubicin, daunorubicin, detorubicin, carminomycin, idarubicin, epirubicin, esorubicin, pirarucibin (THP) and AD-32. More preferably, said anthracycline is daunorubicin, idarubicin, or carminomycin. Yet more preferably said compound of formula (II) is daunorubicin.

The first step of said methods for the preparation of anthracycline-peptide conjugate of formula (I), consist of halogenating the anthracycline of formula (II) at the 14 position. Said halogenation step results in compound of formula (IIa), wherein R¹⁰ represents a halogen and R³, R⁴, R⁵, R⁶, R⁸ and R⁹ have the same meaning as that defined above. According to an embodiment of the present invention, R¹⁰ is Br. According to another embodiment of the present invention, R¹⁰ is Cl. According to yet another embodiment of the present invention, the compound of formula (IIa) consists of a mixture comprising R¹⁰=C¹ and R¹⁰=Br in a ratio of 1/1.

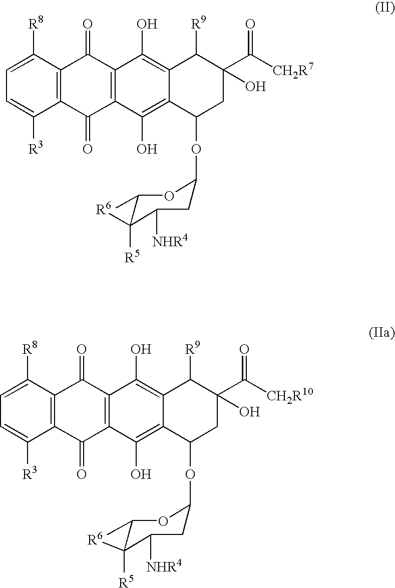

The halogenating agent is preferably the molecular or atomic halogen. The term halogen or halo includes fluoro, chloro, bromo and iodo. According to a preferred embodiment, the halogenation is done with bromine. In general, this halogenation step takes place at a temperature of between 0° C. and 100° C., for example in the region of a point between 0° C. and 50° C., and preferably between 0° C. and 20° C. Generally, the halogenation reaction may be performed in a suitable solvent, such as for example dioxane or chlorinated or simply polar solvents or in a mixture of such solvents. For example, said halogenation may be performed in a mixture of dioxane and methanol.

Said halogenation is preferably done simultaneously with a ketalization step of the 13-ketone of anthracycline of formula (II) in order to protect said ketone function. The ketalization step may be conducted in any suitable manner, but is preferably undertaken by reacting the anthracycline of formula (II) with an alcohol.

Any suitable alcohol may be used in the reaction. Such alcohol should further be provided in excess with respect to the carbonyl groups being ketalized, such as to favor the formation of the ketal. A preferred alcohol for this reaction is methanol. Various orthoesters are suitable for use in the foregoing reaction, the orthoesters functioning to chemically remove the water from the reaction and drive the reaction to completion. Orthoformate esters are advantageously utilized because they provide high yields. Preferred orthoformate esters include triisobutyl orthoformate, triisopropyl orthoformate and triethyl orthoformate, with trimethyl orthoformate being most preferred.

Conversion of the ketal back to the ketone, is accomplished by treatment with aqueous acids. In a preferred embodiment, said aqueous acid is hydrobromic acid.

The next step in said method consists of condensing said halogenated anthracycline of formula (IIa) with the thiol moiety of a peptide of formula (III) according to two alternative routes:
the first route consists of reacting compound of formula (IIa) with a suitable linker of formula (IV) prior to reaction with the peptide of formula (III);
the second route consists of reacting compound of formula (IIa) directly with the peptide of formula (III) thereby obtaining compound of formula (I) wherein L is absent, represented herein by the formula (Ia).

The first route consists of reacting the halogenated anthracycline of formula (IIa) with a linker of formula (IV), thereby producing compound of formula (V); 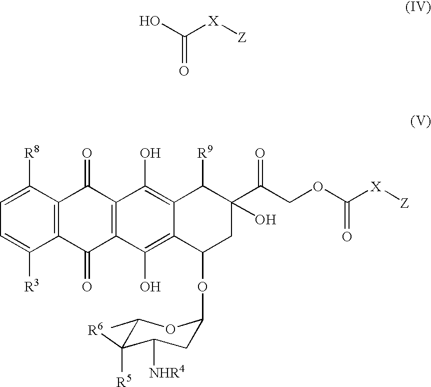
wherein X represents a bivalent radical selected from the group comprising an alkyl, an aralkyl, an alkenyl, a cycloalkyl and an aryl radical, Z represents a functional group capable of reacting with a thiol and R³, R⁴, R⁵, R⁶, R⁸ and R⁹ have the same meaning as that defined above.

In a preferred embodiment, the linker of formula (IV) has a functional group Z which is selected from the group comprising α,β-unsaturated carbonyl, carboxy, carbamoyl and imidyl radical. More preferably, said functional group Z is a maleimidyl radical. In an embodiment, X is a C₁₋₈ alkyl group. In a preferred embodiment, X is a C₁₋₄ alkyl group. According to a more preferred embodiment, X is selected from the group comprising methyl, ethyl, propyl and butyl. Yet, more preferably X is propyl.

According to an embodiment, the linker of formula (IV) is selected from the group comprising 2-chloro-5-maleimidobenzoic acid, 3-maleimidobenzoic acid, 3-maleimidopropionic acid, 4-maleimidosalicylic acid, 6-maleimidohexanoic acid, beta-maleimidopropionic acid, epsilon-maleimidocaproic acid and gamma-maleimidobutyric acid-, or the salts thereof. According to a preferred embodiment, said linker of formula (IV) is maleimidobutyric acid such as for example gamma-maleimidobutyric acid or the salts thereof. In an embodiment of the present invention said linker of formula (IV) is selected from the group comprising sodium maleimidobutyrate and potassium maleimidobutyrate.

The next step in said process consists of coupling said compound of formula (V) with the thiol moiety of a peptide of formula (III) resulting in the compound of formula (I), wherein L represents a linker arm of the formula R—X—Y—, wherein R is —O—C(═O)—, Y is the product of Z upon reaction with the thiol moiety of compound of formula (III) and X, R¹, R², R³, R⁴, R⁵, R⁶, R⁸ and R⁹ have the same meaning as that defined above. According to an embodiment, said peptide is non-oxidized.

Said coupling reaction may be performed in a suitable solvent, non limiting examples of which comprises oxygen free water and DMF.

Said peptide of formula (III) may contain one or several cysteine residues. Cysteine residues provide for the attachment of the linker to the peptide. The use of a cysteine residue for the coupling enhances the selectivity of the coupling. Cysteine residue(s) may be located at either end of the peptide or be internal to the peptide chain, provided that attachment at this site does not interfere with the structure and the properties of the peptide. Irrespective of the cysteine amount, it is preferred that one cysteine residue be located at the N- or C-terminal end. Examples of suitable peptides have a cysteine residue at the C-terminal end of said peptide.

Said peptide of formula (III) may be chemically synthesized or produced by recombinant means. Either method can be achieved conventionally. Said peptide includes those with unnatural or non-amino acids. These peptides, which would be made by chemical synthesis, include those with modified amino acids or other moieties in place of amino acids. Such other moieties include but are not limited to fluorine, chlorine, and organic compounds such as alcohols, organic ring structures and hydroxyacids. Amino acids or peptides in the D-orientation can also be used, as can peptides in the reverse orientation. Peptidomimetics and peptoids are also encompassed in the present invention, wherein “peptidomimetic” as used herein represents a molecule which mimics the biological activity of a peptide, by substantially duplicating the pharmacologically relevant portion of the conformation of the peptide, but is not a peptide. The term “peptoid” as used herein represents an analogue of a peptide in which one or more of the peptide bonds are replaced by pseudopeptide bonds, which may be the same or different. Such pseudopeptide bonds may be carba Ψ (CH₂—CH₂); depsi Ψ (C(═O)O); hydroxyethylene Ψ (CHOH—CH₂); ketomethylene Ψ (CO—CH₂); methylene-oxy CH₂—O—; reduced CH₂—NH; thiomethylene CH₂—S—; thiopeptide CS—NH; N-modified —NRCO—; retro-inverso —CO—NH—. A single peptoid molecule may include more than one kind of pseudopeptide bond. It may also include normal peptide bonds.

According to a preferred embodiment said peptide of formula (III) contains from 1 to 100 amino acids, preferably from 10 to 50, more preferably from 10 to 40, yet more preferably from 10 to 30 amino acids.

Examples of suitable peptide of formula (III) include but are not limited to those that contain amino acids selected from the group comprising non-polar amino acid, positively charged amino acid, polar uncharged amino acid and negatively charged amino acid. For example, said peptide of formula (III) may contain at least 3 positively charged amino adds.

Other suitable examples of peptide of formula (III) include but are not limited to those that contain from 45% to 90% positively charged amino acids; preferably from 45% to 80%, more preferably from 45% to 70%, most preferably from 45% to 60%.

For example said peptide of formula (III) may consist of the following sequence of amino acid Cys N N P N P B P P N P P P P P A P N B P B N P B P B P P B B N, wherein ‘N’ is a non-polar amino acid, ‘B’ is positively charged amino acid, ‘P’ is a polar uncharged amino acid and ‘A’ is an negatively charged amino acid.

As used herein non-polar amino acids are A, I, L, M, F, P, W and V. Polar uncharged amino acids are N, C, Q, G, S, T and Y. Positively charged amino acids are R, H and K. Negatively charged amino acids are D and E.

In an embodiment of the present invention, the peptide may be a peptide able to carry the anthracycline conjugate of the invention inside the cells, which could allow overcoming anticancer drug resistance problems. Said peptide may facilitate internalization of the anthracycline conjugate in the cytoplasm through its interaction with the cell membrane.

Examples of compounds prepared by the present method include but are not limited to compound of formula (Id), wherein R¹ and R² have the same meaning as that defined above and n is a number ranging from 2 to 10, such as for example compounds of formula (Ic).

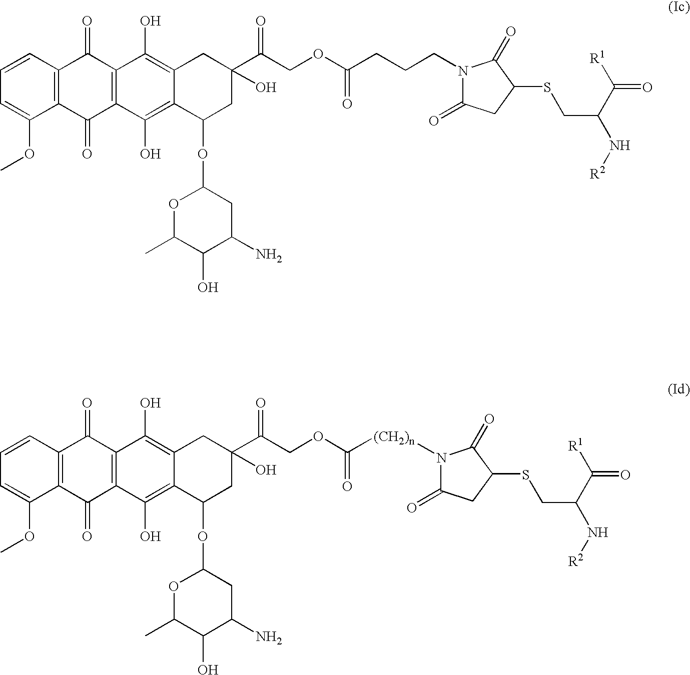

The second route consists of reacting the halogenated anthracycline of formula (IIa) with the thiol moiety of the peptide of formula (III) as described above, thereby obtaining compound of formulae (I) wherein L is absent as represented in formula (Ia).

Said reaction may be performed in the presence of a suitable solvent such as methanol. The reaction is suitably performed under basic condition such as pH 10 or above. The reaction condition can be rendered basic by the addition of a suitable base such as potassium carbonate.

One skilled in the art understands that in the synthesis of compounds of the invention, one may need to protect various reactive functionalities on the starting compounds and intermediates while a desired reaction is carried out on other portions of the molecule. After the desired reactions are complete, or at any desired time, normally such protecting groups will be removed by, for example, hydrolytic or hydrogenolytic means. Such protection and deprotection steps are conventional in organic chemistry (Protective Groups in Organic Chemistry, McOmie, ed., Plenum Press, NY, N.Y. (1973); and, Protective Groups in Organic Synthesis, Greene, ed., John Wiley & Sons, NY, N.Y. (1981)).

In the method described herein, the compounds and intermediates may be further purified according to methodologies generally known in the art such as, for example, extraction, crystallization, trituration and chromatography.

Another aspect of the present invention relates to intermediates and compounds obtained by the above-described methods.

More in particular, the present invention relates to compounds having the formula (Ia), wherein R³ represents OCH₃, OH or H, R⁴ represents H or COCF₃, R⁵ represents OH, O-tetrahydropyranyl or H, R⁶ represents OH or H, R⁸ represents OH or H, R⁹ represents OH or H; R¹ represents OH, NH₂ or NH-peptide and R² represents H or —CO-peptide.

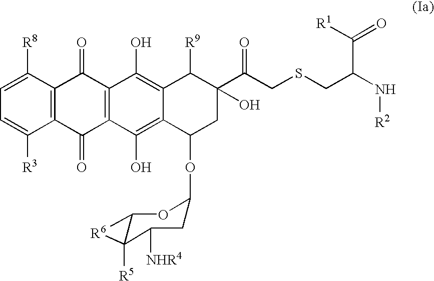

According to another embodiment, the present invention relates to compounds of formula (Ia) wherein R³ represents OCH₃, OH or H, R4 is H, R⁵ represents OH, O-tetrahydropyranyl or H, R⁶ represents OH or H, R⁸ is H, R⁹ is H; R¹ represents OH, NH₂ or NH-peptide and R² represents H or —CO-peptide.

According to yet another embodiment, the present invention relates to compounds of formula (Ia) wherein R³ represents OCH₃, OH or H, R⁴ is H, R⁵ is OH, R⁶ is H, R⁸ is H, R⁹ represents H; R¹ represents OH, NH₂ or NH-peptide and R² represents H or CO-peptide.

According to a further embodiment, the present invention relates to compound of formula (Ib), wherein R¹ and R² have the same meaning as that defined above.

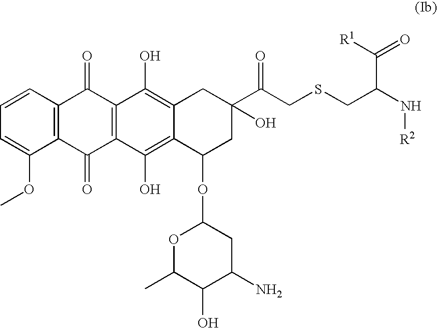

Said new compound according to the invention may contain from 1 to 100 amino acids, preferably from 10 to 50, more preferably from 10 to 30 amino acids. According to an embodiment, said compound may contain at least 3 positively charged amino acids.

The compounds according to the invention may contain amino acids selected from the group comprising non-polar amino acid, polar uncharged amino acid and positively or negatively charged amino acid.

For example said new compound may contain from 45% to 90% positively charged amino acids, preferably from 45% to 80%, more preferably from 45% to 70%, most preferably from 45% to 60%.

The compounds according to the invention may contain the following sequence of amino acid Cys N N P N P B P P N P P P P P A P N B P B N P B P B P P B B N, wherein ‘N’ is a non-polar amino acid, ‘B’ is a positively charged amino acid, ‘P’ is a polar uncharged amino acid and ‘A’ is an negatively charged amino acid.

The present invention also encompasses alternative forms of said compounds such as pharmaceutically acceptable salts, solvates, hydrates, and the like. The pharmaceutically acceptable salts of the compounds of this invention include the conventional non-toxic salts of the compounds of this invention as formed, e.g., from non-toxic inorganic or organic acids. For example, such conventional non-toxic salts include those derived from inorganic acids such as hydrochloric, hydrobromic, sulfuric, sulfamic, phosphoric, nitric and the like: and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, trifluoroacetic and the like.

The new compounds or pharmaceutical compositions thereof are useful as medicament and more particularly as medicament for the treatment of cancer and drug resistant cancer. The intermediates according to the invention are also useful as a precursor in the preparation of antitumor agent.

Said new compounds conjugates of the invention or pharmaceutically acceptable salt thereof, can be administered to a patient in the form of a pharmaceutical composition comprising a pharmaceutical carrier and a therapeutically effective amount of said above-described compounds. Said composition may further include thickeners, diluents, buffers, preservatives, surface active agents, liposomes, or lipid formulations, and the like. Said pharmaceutical composition may also include one or more additional active ingredients such as other chemotherapy agents, antimicrobial agents, anti-inflammatory agents, anesthetics, and the like.

Said pharmaceutical composition may be administered in a number of ways depending on whether local or systemic treatment is desired, and on the area to be treated. Administration may be topically including on the skin, ophthalmically, vaginally, rectally, intranasally, orally, by inhalation, or parenterally, for example by intravenous drip, subcutaneous, intratumor, intraperitoneal, intralymphatic or intramuscular injection. The preferred mode of administration is parenterally.

With formulations for topical administration may include ointments, lotions, creams, gels, drops, suppositories, sprays, liquids and powders. Conventional pharmaceutical carriers, aqueous, powder or oily bases, thickeners, and the like may be necessary or desirable. Compositions for oral administration include powders or granules, suspensions or solutions in water or non-aqueous media, capsules, or tablets. Thickeners, flavorings, diluents, emulsifiers, dispersing aids or binders may be desirable. Formulations for parenteral administration may include sterile aqueous solutions optionally containing buffers, liposomes, diluents and other suitable additives.

The “therapeutically effective amount” of said above-described new compounds relates to the amount or quantity of compound according to the invention which is sufficient to elicit the required or desired therapeutic response, or in other words, the amount which is sufficient to elicit an appreciable biological response when administered to a patient. Dosing is dependent on the severity and responsiveness of the condition to be treated, with course of treatment lasting from several days to several months or until a cure is effected or a diminution of disease state is achieved. Optimal dosing schedules and dosing amounts can be calculated based on the chemotherapy agent alone. The conjugated compound or the co-administered compound can then be compared to the chemotherapy agent alone, and the dosages can be adjusted accordingly. For instance, optimal dosages are generally 10× below the lethal dose. Optimal dosing schedules can also be calculated from measurements of drug accumulation in the body. Persons of ordinary skill in the art can easily determine optimum dosages, and dosing methods.

The new compounds or pharmaceutical compositions thereof are useful as medicament and, more particularly as medicament for the treatment of cancer and drug resistant cancer. Said new compounds are therefore useful as antitumor agent, and may be used for the preparation of medicament for treating cancer.

The present invention furthermore relates to a method of treating a patient suffering from cancer, wherein an anthracycline-peptide conjugate as described above is administered to the patient.

The following examples are meant to be illustrative of the present invention. These examples are presented to exemplify the invention and are not to be construed as limiting the invention's scope.

### EXAMPLE 1

### Synthesis of Anthracycline-Peptide Conjugates of Formula (Ic) Starting from Daunorubicin as Illustrated in Scheme 1

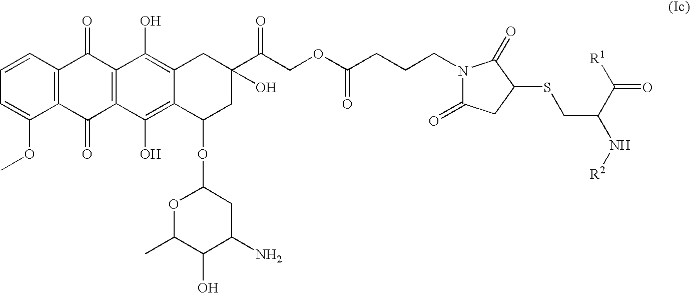

14-Bromo-daunorubicin via 14-bromo-13-dimethylketal-daunorubicin: Daunorubicin.HCl (1.065 mmol) is dissolved in a mixture of dry methanol (6 ml) and dry dioxane (6 ml). Trimethyl orthoformate (4.896 mmol, 4.6 eq.) is then added followed by bromine (1.404 mmol, 1.31 eq.). The mixture is stirred one hour at 15° C. under argon. Propylene oxide (2.748 mmol, 2.57 eq.) is then added, and after 30 minutes at 4° C., isopropylether (65 ml) is added. A precipitate of 14-bromo-13-dimethylketal-daunorubicin immediately forms and is recovered by centrifugation (5 minutes, 1000 g). This precipitate is further washed with a second portion of isopropylether (8.4 ml) and dried under argon.

14-Bromo-13-dimethylketal-daunorubicin is suspended in acetone (22.8 ml) and a 0.25 M HBr aqueous solution (22 ml) is added. The solution is stirred 45 hours at room temperature under argon, then diluted with water (27 ml) and extracted with chloroform (2×65 ml). Saturated NaCl (6 ml) is added to the aqueous layer that is then extracted with n-butanol (24 ml for each extraction step) until it becomes colorless. The organic layers are combined and solvent is evaporated (high vacuum pump, 30-35° C.) until precipitation of 14-bromo-daunorubicin. n-Hexane (50 ml) is added, and the precipitate is recovered by filtration, washed with n-hexane and dried (yield, 80%).

Doxorubicin-14-maleimidobutyrate: 14-bromo-daunorubicin (0.851 mmol) is suspended in acetone (80 ml) and sodium maleimydobutyrate (4.91 mmol, 5.77 eq.) is added. The mixture is refluxed 2 hours, cooled down to room temperature, and filtered on quantitative paper. The precipitate is washed with acetone and the combined filtrates are evaporated (bath: 30° C.). The residue is dissolved in water and incubated with an anion-exchange resin (Amberlite IRA-402Cl) in order to remove excess maleimidobutyrate. Alternatively, a YMC silica gel may also be used. After lyophilization, doxorubicin-14-maleimidobutyrate is obtained in 79% yield.

Doxorubicin-peptide conjugate. Doxorubicin-14-maleimidobutyrate (0.076 mmol) is dissolved in DMF (5 ml) and the non-oxidized peptide (0.7 eq., 0.053 mmol taking into account actual peptide content) previously dissolved in dimethylformamide (DMF, 5 ml) is added. After a 3-hour to 24-hour stirring (depending on the peptide) at room temperature and under argon, water (10 ml) is added and the solution is extracted with dichloromethane (DCM, 6×20 ml). The aqueous layer is lyophilized to give the doxorubicin-peptide conjugate. The reaction can also be done in water (9 ml). After stirring, the mixture is extracted with DCM/DMF: 9/1 (25×9 ml) then with DCM (6×9 ml). The doxorubicin-peptide conjugates can be purified by reverse phase high-pressure liquid chromatography. For example, a 250×21.2 mm, 10μ Luna column (Phenomenex) can be used with 0.1% trifluoroacetic acid in water and 0.1% trifluoroacetic acid (TFA) in acetonitrile as solvents. A 20-40% acetonitrile gradient in 70 minutes (with a flow rate of 6 ml/min) allows an appropriate separation. A maximum of the acetonitrile and trifluoroacetic acid content is removed from fractions containing the conjugate by bubbling with nitrogen or argon prior to lyophilization. 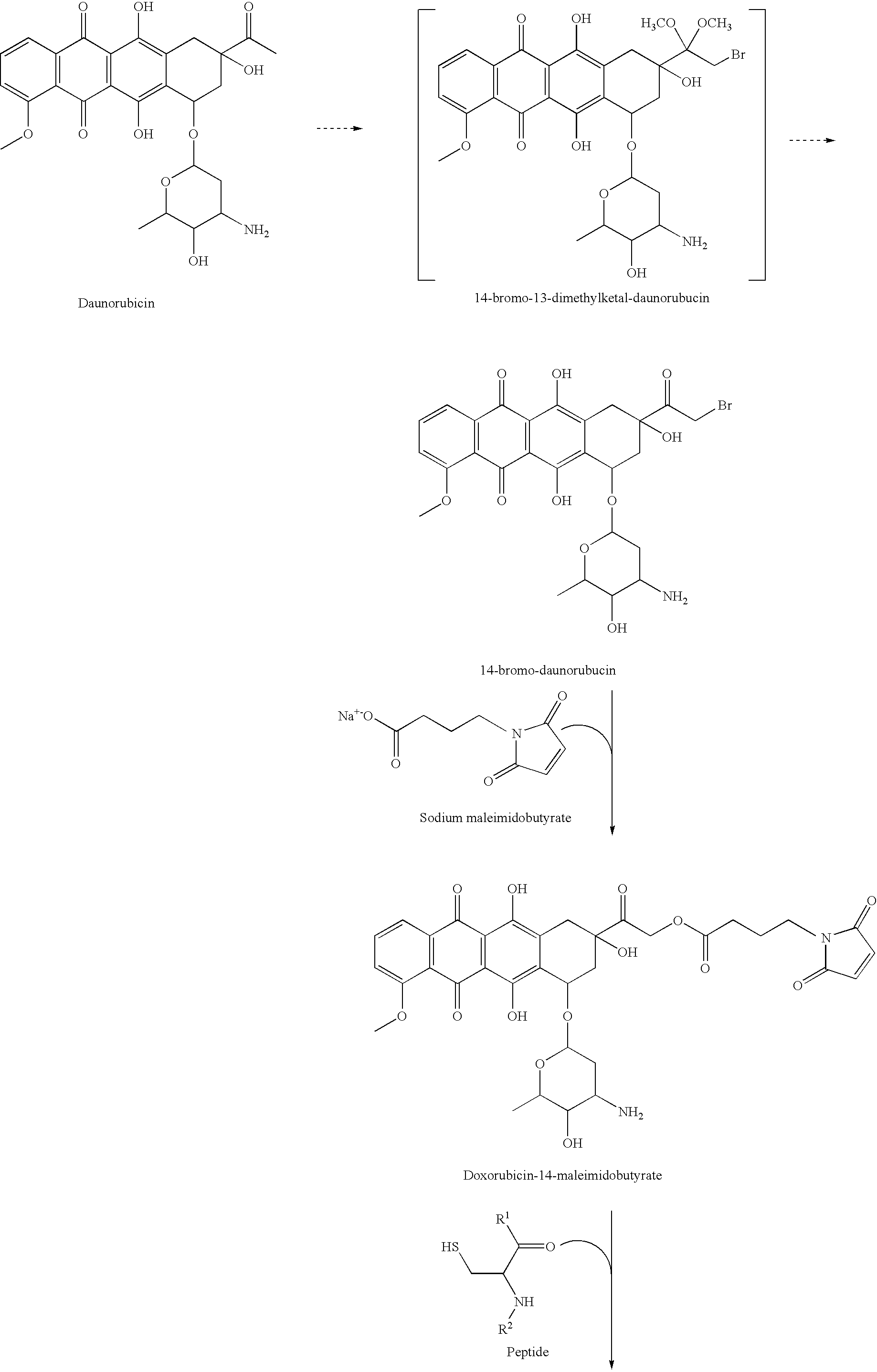 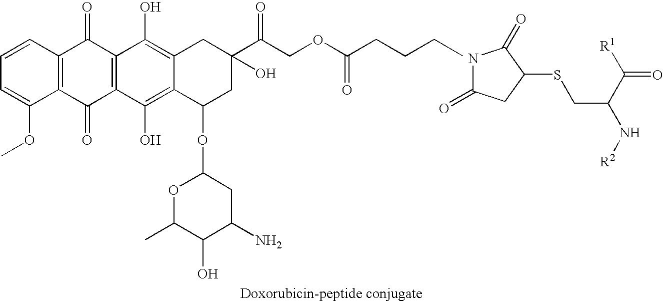
Scheme 1. Synthesis of doxorubicin-peptide conjugates starting from daunorubicin wherein R¹=—OH, —NH₂, or —NH-peptide; R²=—H or —CO-peptide.

### EXAMPLE 2

### Synthesis of Anthracycline-Peptide Conjugates of Formula (Ib) as Illustrated in Scheme 2

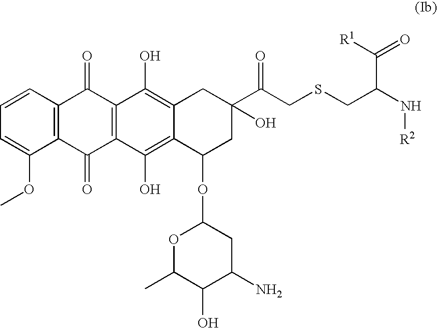

14-Bromo-daunorubicin (0.350 mmol) is dissolved in dry methanol (12 ml) in a round-bottom flask and peptide (0.85 eq. taking peptide content into account) is added followed by K₂CO₃ (1.3 eq.) (pH must reach 10, if not, potassium carbonate is added). The reaction mixture is stirred for 30 to 90 min (depending on the peptide) under argon and protected from light. Work-up is initiated by the addition of a 0.5 M Tris-HCl buffer pH 9 ( 1/10 of methanol volume) and extractions with chloroform (6×1 volume) until the organic layer becomes colorless. The aqueous layer is then loaded on a YMC ODS-A solid-phase extraction resin (5 g/100 mg of crude compound) preconditioned with methanol and water in a glass frit. After washes with 0.1% TFA in water, the conjugate is recovered by elution with methanol. Methanol is evaporated, the residue is dissolved in water and the resulting solution is then lyophilized to yield the crude thioether conjugate. 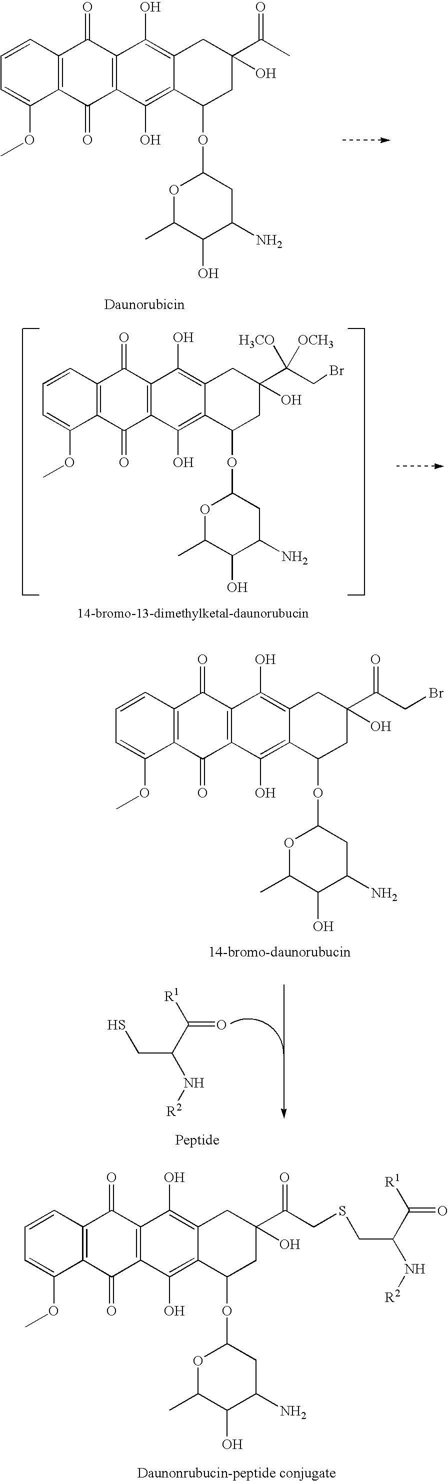
Scheme 2. Synthesis of daunorubicin-peptide conjugates starting from daunorubicin wherein R¹=—OH, —NH₂, or —NH-peptide; R²=—H or —CO-peptide.

### EXAMPLE 3

### Multigram Scale Synthesis of Anthracycline-Peptide Conjugates of Formula (Ic) (Scale-Up Factor=30, Scheme 3)

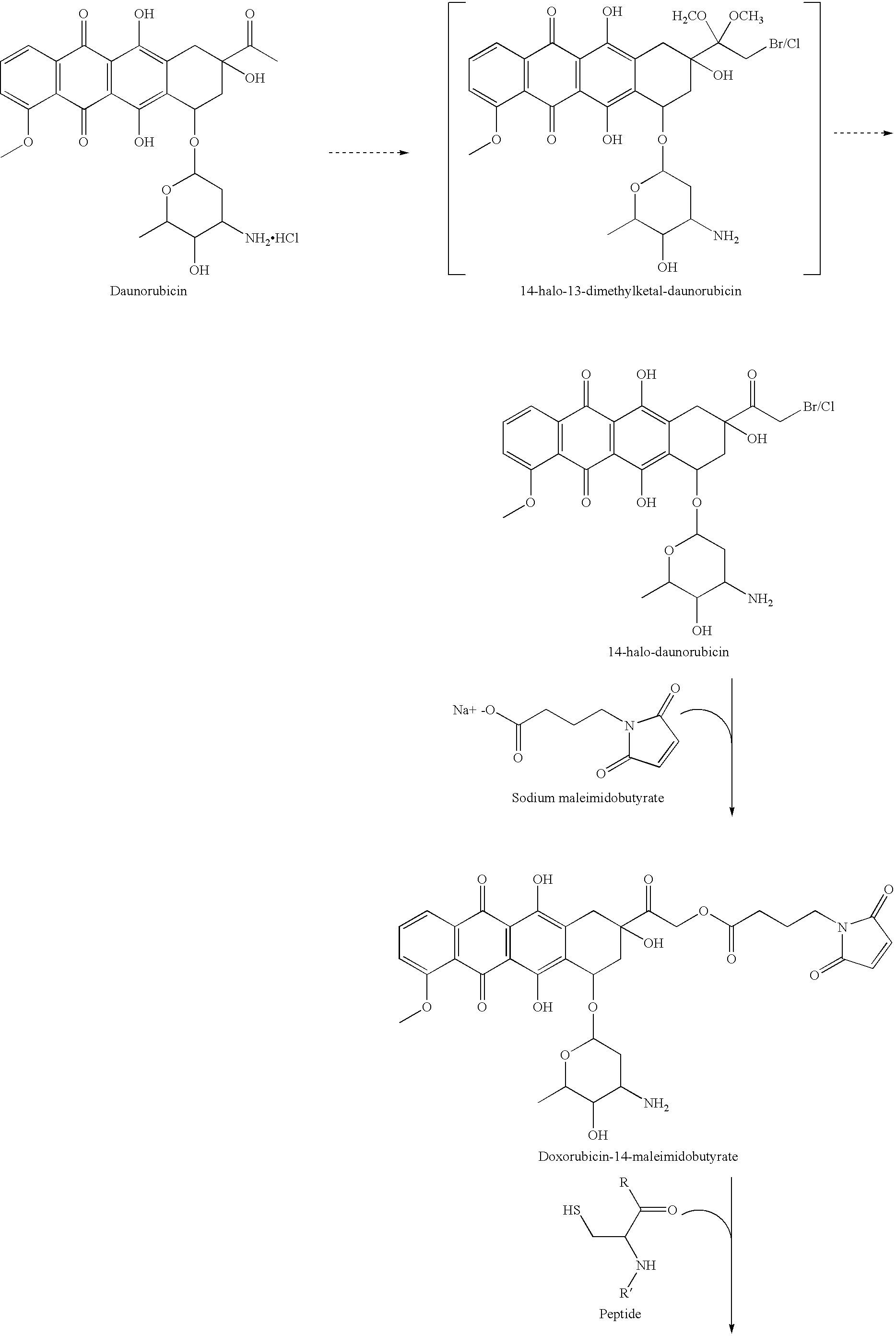 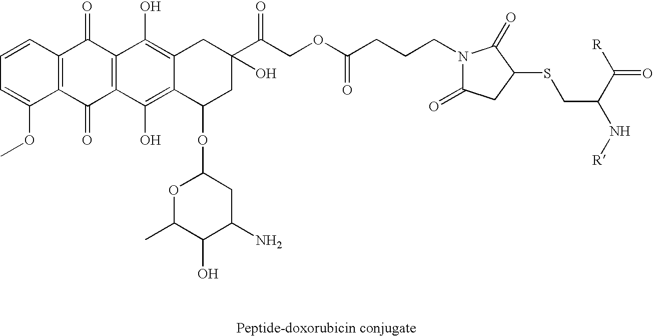 [0073] Scheme 3. Synthesis of doxorubicin-peptide conjugates starting from daunorubicin wherein R¹=—OH, —NH₂, or —NH-peptide; R²=—H or —CO-peptide.

The different steps of the synthesis of anthracycline-peptide conjugates of formula (Ic) starting from daunorubicin (scheme 1 or 3) were improved and adapted to a multigram scale (30 mmol of starting daunorubicin; previously 1 mmol).

Synthesis of 14-Bromo-daunorubicin via 14-bromo-13-dimethylketal-daunorubicin: Daunorubicin.HCl (30.0 mmol) is dissolved in a mixture of dry methanol (207 ml) and dry dioxane (207 ml). Trimethyl orthoformate (138.5 mmol, 4.6 eq.) is then added and the mixture is stirred at room temperature for 5 min. The solution is cooled to 12° C. and bromine (51.5 mmol, 1.31 eq.) is added over 2 min. The mixture is stirred at 12° C. under argon during two hours, then cooled down to 2° C. prior to the addition of propylene oxide (78 mmol, 2.57 eq.). After 75 minutes at 2° C., isopropylether (1740 ml) is added, a precipitate of 14-bromo-13-dimethylketal-daunorubicin immediately forms and is recovered by filtration through quantitative filter paper. This precipitate is further washed with a second portion of isopropylether (540 ml).

The 14-Bromo-13-dimethylketal-daunorubicin is then dissolved in a mixture of acetone (690 ml) and 0.25 M aqueous HBr (600 ml). The solution is stirred 66 hours at room temperature under argon, then diluted with water (750 ml) and extracted with chloroform (3×750 ml). Saturated NaCl (150 ml) is added to the aqueous layer that is then extracted with n-butanol (2×1.5 l, 2×0.75 l) until it becomes colorless. The organic layers are combined and solvent is evaporated (high vacuum pump, 3035° C.) to a volume of approximately 300 ml. n-Hexane (2 l) is added, and the precipitate is recovered by filtration, washed with n-hexane and dried (yield, 71%). The resulting red solid was determined to be a mixture of 14-Bromo-daunorubicin and 14-Chloro-daunorubicin (approximately 1/1) by LC-MS analysis and by proton NMR spectrometry.

Halogen exchange can be prevented by using saturated sodium bromide instead of sodium chloride during the extraction step. Table 1 presents the HPLC peak area of halo-daunorubicin species found in different syntheses.

TABLE 1
Percentage of halo-daunorubicin species.

| | 14-Cl-Dnr (% peak area) | 14-Br-Dnr (% peak area) |
|---|---|---|
| Acetal intermediate | 9 | 72 |
| Addition of NaCl | 55 | 30 |
| Addition of NaBr | 15 | 75 |

Analysis of the acetal intermediate showed the presence of 14-Chloro-daunorubicin presumably arising because the starting material was a hydrochloride salt. Subsequent addition of sodium chloride solution led to a large amount of halogen exchange. It is possible to form 14-Bromo-daunorubicin alone by avoiding the presence of chloride ions.

Preparation of doxorubicin-14-maleimidobutyrate with different preparations of Br/Cl-daunorubicin: A sequence of reactions were carried out using halo-daunorubicin species containing different proportions of the 14-Cl-daunorubicin and 14-Br-daunorubicin compounds in order to obtain both the best yield and purity for the esterification reaction. Each of these different halo-daunorubicin preparations were reacted with sodium maleimidobutyrate obtained as described below. The results are summarized in Table 2.

TABLE 2
Results of the coupling of 14-halo-daunorubicin with sodium
maleimidobutyrate.

| Br-daunorubicin/Cl-daunorubicin | Yield (%) |
|---|---|
| 9/1 | 28 |
| 3/1 | approximately 30 |
| 1/1 | 69 |
| 1/6 | 69^{a} |
| 0/1 | 62^{a} |

^{a}24% or more of acetone adduct and a large amount of starting material

It is clear from these studies that the coupling reaction works best in the presence of significant amounts of the 14-Cl-daunorubicin species. Use of the essentially pure 14-chloro compound was not ideal, however, since the lower reactivity of this species gave rise to significant amounts of unreacted starting material. The best overall result was obtained with an approximate 1/1 14-Chloro to 14-Bromo-daunorubicin ratio.

Synthesis of sodium maleimidobutyrate: Sodium hydrogenocarbonate (100 mmol) is dissolved in water in a 1-l volumetric flask to produce a 0.1 M solution. A portion of this solution (435 ml, 43.5 mmol) is added slowly via a dropping funnel to a stirred suspension of 4-maleimidobutyric acid (8.014 g, 43.75 mmol) in water (80 ml). The resulting solution is stirred for 20 min and water is evaporated in vacuo at 30° C. (water bath temperature) before final drying on a freeze-drying unit. The product is obtained as an off-white/slightly pink solid (9.06 g, 100%).

Synthesis of doxorubicin-14-maleimidobutyrate: 14-bromo-daunorubicin/14-chloro-daunorubicin approximately 1/1 (12.6 mmol) is suspended in acetone (1.2 l) and sodium maleimydobutyrate (65.8 mmol, 5.77 eq.) is added. The mixture is refluxed 3 hours under argon, cooled down to room temperature, and filtered on quantitative paper. The precipitate is washed with acetone and the combined filtrates are evaporated (bath: 30° C.). The residue is dissolved in water and incubated with an anion-exchange resin (Amberlite IRA-402Cl) in order to remove excess maleimidobutyrate. It can also be passed through a YMC reverse-phase gel. After lyophilization, doxorubicin-14-maleimidobutyrate is obtained in 69% yield containing one major impurity, formed consecutively to the esterification reaction and identified by mass spectrometry as an acetone adduct of doxorubicin-14-maleimidobutyrate.

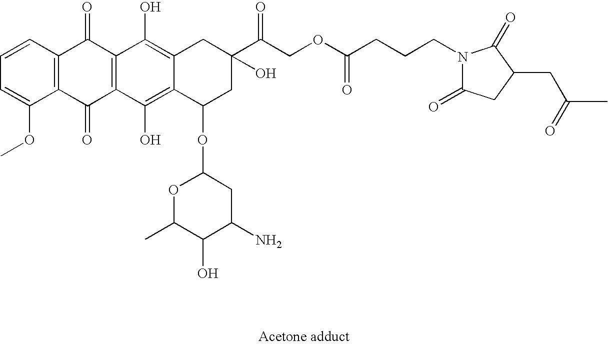

Use of alternative solvents for the esterification: The formation of an acetone adduct as a by-product during the coupling of sodium maleimydobutyrate and the halo-daunorubicin is wasteful of the expensive daunorubicin starting material. Several other solvents were assessed in an attempt to find an alternative coupling medium that would not lead to the formation of the acetone adduct. The ideal solvent needs to be capable of dissolving both of the 14-halo-daunorubicin, starting materials, as well as the ester product, whilst being a poor solvent for sodium maleimydobutyrate and any inorganic by-products of the coupling reaction. Several solvents were tested, as shown in table 2, but none of them proved to be superior or even equal to acetone.

TABLE 2
Alternative solvents for esterification

| Solvent^{a} | T (h) | % ester |
|---|---|---|
| THF | 3 | 8 |
| ACN | 3 | 16 |
| Dioxane | 2 | 18 |
| 1,2-DME | 2 | 20 |
| DMF^{b} | 1 | 0 |
| NMP^{b} | 1 | 0 |
| Butanone | 2 | 20 |

^{a}reaction temperature: 60° C.;
^{b}complete degradation of the mixture

Acetone remains the best solvent to date for the coupling reaction. Although the use of acetone as the solvent leads to the isolation of an impure product, the by-product obtained is not reactive in the next step and can be removed by an extraction step.

The preparation of the approximately 1/1 mixture of 14-Br-daunorubicin and 14-Cl-daunorubicin appears to be a generally robust process; which has worked equally on both small (300 mg) an intermediate (16 g) scales.

Synthesis of doxorubicin-peptide conjugate: Doxorubicin-14-maleimidobutyrate (3.91 mmol) is dissolved in water (160 ml, oxygen-free) and the non-oxidized peptide (0.7 eq., 3.27 mmol taking into account actual peptide content) previously dissolved in oxygen-free water (160 ml) is added. An extra 80 ml of water is added to rinse out the flask. After a 48hour stirring at room temperature and under argon, the resulting solution is extracted with DCM/DMF: 9/1 (30×100 ml) then with DCM (3×100 ml). This conjugate was purified by means of preparative HPLC separation using a Micromass ZMD instrument and a Luna C18(2), 10 μm, 250×21.2 mm semi-preparative column (Phenomenex ref. 00G-4253-P0) with 0.1% TFA in water as solvent A and 0.1% TFA in acetonitrile as solvent B (gradient: 5-30% B in 10 min, 3 min at 30% B, 30-90% B in 1 min, 6 min at 90% B; flow: 20 m/min; loading: 200 mg/run in 1 ml).

Large scale salt exchange of the Doxorubicin-peptide TFA salt: A trifluoroacetic acid (TFA) salt can not be developed and used as a medicine because of the poorly characterized toxicity profile of TFA. That is why a salt exchange method is required to transform the TFA salt in an HCl salt.

Ion-exchange method: Amberlite IRA-410 (Cl) ion-exchange resin (260 g) is mixed in a beaker with 2 M hydrochloric acid (400 ml) and stirred for 30 min. The resulting slurry of resin is then loaded into a 6.5 cm diameter glass chromatography column (height of resin column=11 cm) and the liquid blown through with a slight positive pressure of nitrogen. The packed resin is eluted with water until the eluate has neutral pH (1.2 l water required). Doxorubicin-peptide TFA salt (2.5 g) is dissolved in water (25 ml) and loaded onto the ion-exchange column. The column is then eluted with water, with approximately 35 ml fractions being collected, until the dark red product band has been collected. The process is then repeated, in the manner described above, using a second column of Amberlite JRA-410 (Cl) resin. All of the relevant red-colored, product-containing, fractions from both columns are combined and freeze-dried to give the final HCl salt product as a red colored solid (3.72 g, 90%). Residual TFA analysis:<0.1%.

### EXAMPLE 4

### Synthesis of 14-Bromo-daunorubicin and its Maleimide Derivative Without Acetal Isolation (Scheme 4)

Daunorubicin.HCl (7.9 mmol) is dissolved in a mixture of dry methanol (35.1 g) and dry dioxane (38 g). Trimethyl orthoformate (36.7 mmol, 4.6 eq.) is then added and the mixture is stirred at room temperature for 10 min. The solution is cooled to 12° C. and bromine (13.7 mmol, 1.31 eq.) is added over 5 min. The mixture is stirred two hours at 20° C. Propylene oxide (20.5 mmol, 2.57 eq.) is added at 2° C. over 10 min, and after 75 minutes at 2° C., the mixture is warmed to 20° C. Acetone (100 g) and a 7% (w/w) HBr aqueous solution (6.4 g HBr 48% and 37.6 g water) are added. The solution is stirred 35 hours at 20° C., then diluted with water (120 g) and extracted with chloroform (2×150 g). A solution of NaCl (46.8 g in 133.2 g water) is added to the aqueous layer that is then extracted with n-butanol (2×100 g) until it becomes colorless. The organic layers are combined and solvent is evaporated (high vacuum pump, 30-35° C.) to a volume of approximately 102 g. n-Hexane (80 g) is added, and the precipitate is recovered by filtration, washed with n-hexane (100 g) and dried (yield, 98%). The compound is a mixture of Cl- and Br-Daunorubicin in approximately 1/1 ratio.

Synthesis of potassium maleimidobutyrate with tBuOK: To a stirred solution of 4-maleimidobutyric acid (5 g, 0.0273 mmol) in THF (65.2 g), a solution of tBuOK in THF (23.5 g, 0.95 eq.) is added, at room temperature, over 15 min. The resulting suspension is kept at 4° C. before use without further treatment and isolation (yield 100%). 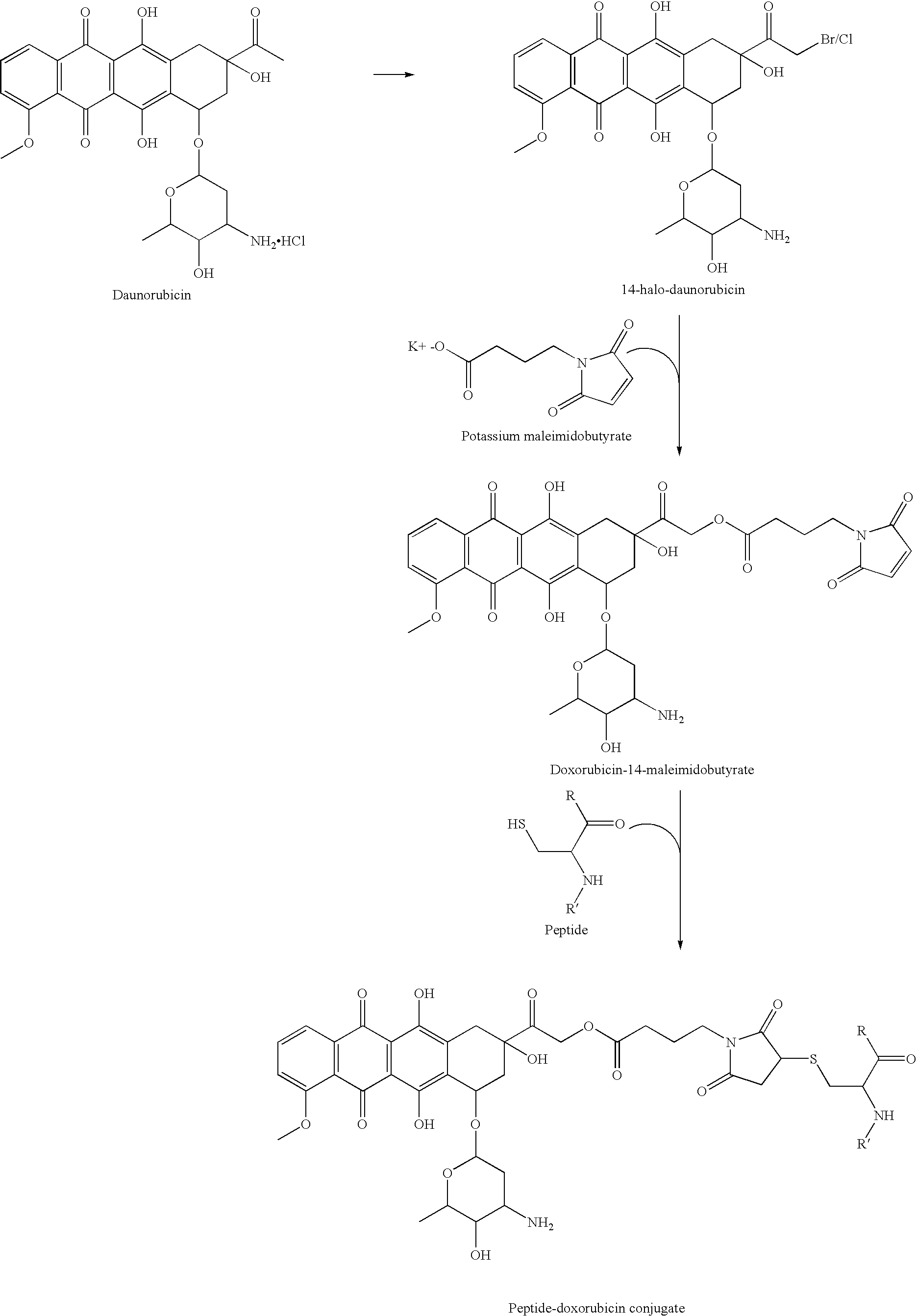
Scheme 4. Synthesis of doxorubicin-peptide conjugates starting from daunorubicin wherein R¹=—OH, —NH₂, or —NH-peptide; R²=—H or —CO-peptide.

Continuous work to improve the process and its scalability in compliance with GMPs led to the following further improvements [results obtained at multigram scale (8 mmol of Daunorubicin in each case)]:

| Compound | Improvement |
|---|---|
| 14-Halo-13- | Volumes of methanol and dioxane are reduced by |
| dimethylketal- | 20%. The non-isolated-product (acetal) is used |
| daunorubicin | directly (scheme 4) |
| 14-Bromo- | Reduction of solvent volumes (1.5x for acetone, |
| daunorubicin | 3.7x for HBr) was validated, reaction time can be |
| | reduced to 35 hours instead of 66, the number (and |
| | volume) of extraction steps (2 vs 3) was reduced as |
| | well. No use of high vacuum pump anymore. |
| | Yield could be increased by about 25%. |
| Potassium | Potassium maleimidobutyrate can advantageously |
| maleimidobutyrate | be prepared in tetrahydrofuran (THF) with |
| | potassium tertiobutylate (tBuOK). |
| Doxorubicin-14- | The potassium maleimidobutyrate suspension (3 eq. |
| maleimidobutyrate | instead of 6 used previously) is used as such (no |
| | product isolation). This permit to avoid the |
| | lyophilization step and decreases the formation of |
| | acetone adduct. |

In summary, these examples have shown that the anthracycline-peptide conjugates of the present invention can be prepared through easy to implement methods comprising reduced number of steps. Furthermore, said conjugates can be prepared cheaply from readily available starting materials and reagents.

These synthetic methods according to the invention have the advantage of being the most interesting route to the synthesis of such compounds for different reasons. First, there are no protection/deprotection steps involved in the synthesis of these compounds. Second, the intermediate compounds can be produced in high quantities and are stable several weeks (bromodaunorubicin in a dessicator at room temperature, doxorubicin-14-maleimidobutyrate at −20° C.). It should be noticed that the stability of bromodaunorubicin at room temperature in a dessicator is something that was unexpected. As a matter of fact, this compound is known as particularly unstable in most conditions (see EP 0 295 119 B1). The intermediate compounds prepared according to the methods of the invention, have a good stability, which make them useful intermediates for the anthracycline-peptide conjugates production purposes.

Moreover, in the cases of the preparation of the anthracycline-peptide conjugates of formula (Ia) and (Ib), the reaction of haloanthracyclines with the thiol moiety of the peptides used in the present method proved to work very well despite the size of the peptide (MW>2500).

1. Method for the preparation of a compound of formula (I) or pharmaceutically acceptable salts thereof and intermediates thereof, comprising the steps of: 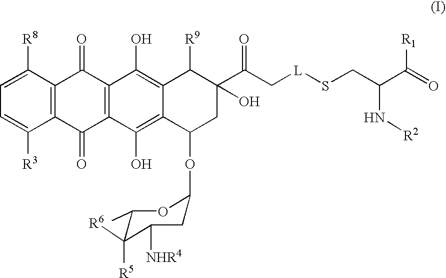
a) halogenating a compound of formula (II), resulting in compound of formula (IIa), 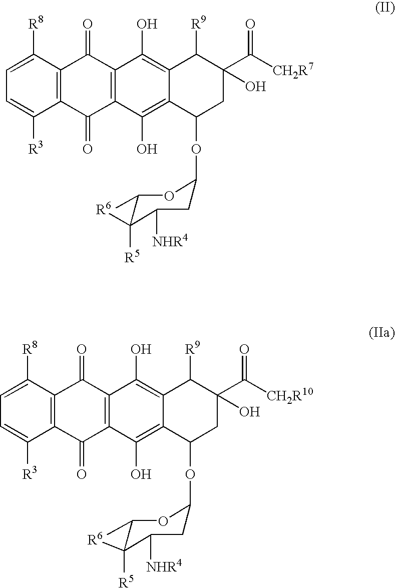
b) reacting a compound of formula (IIa) at its 14 position with the thiol moiety of a peptide of formula (III), optionally in the presence of a suitable linker, to obtain said compound of formula (I), 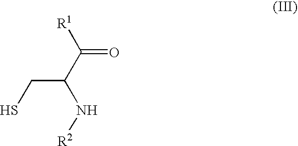
wherein R¹ represents OH, NH₂ or NH-peptide; R² represents H or —CO-peptide; R³ represents OCH₃, OH or H; R⁴ represents H, or COCF₃; R⁵ represents OH, O-tetrahydropyranyl or H; R⁶ represents OH or H; R⁷ represents H, OH, OCO(CH₂)₃CH₃ or OCOCH(OC₂H₅)₂; R⁸ represents OH or H; R⁹ represents OH or H; R¹⁰ represents a halogen and S is either directly linked to C or linked through L, wherein L is a suitable linker arm.

2. Method according to claim 1, further comprising the step of
a) halogenating the compound of formula (II), resulting in compound of formula (IIa), 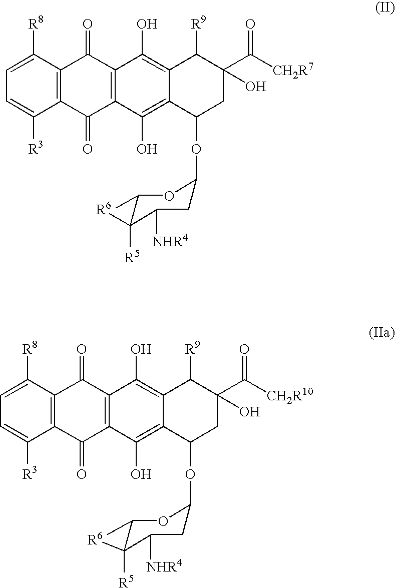
b) reacting said compound of formula (IIa) at its 14 position with a linker of formula (IV) to obtain compound of formula (V), wherein Z is a functional group able to react with a thiol, and X represents a bivalent radical selected from the group consisting of an alkyl, an aralkyl, an alkenyl, a cycloalkyl and an aryl radical 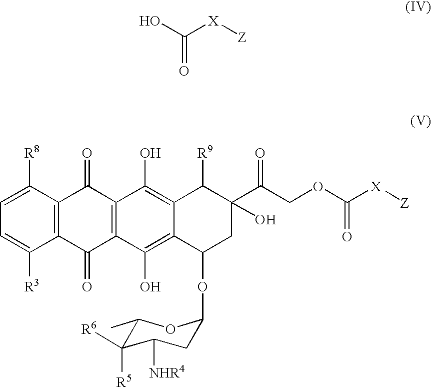
c) coupling said compound of formula (V) with the thiol moiety of a peptide of formula (III) to obtain compound of formula (I), 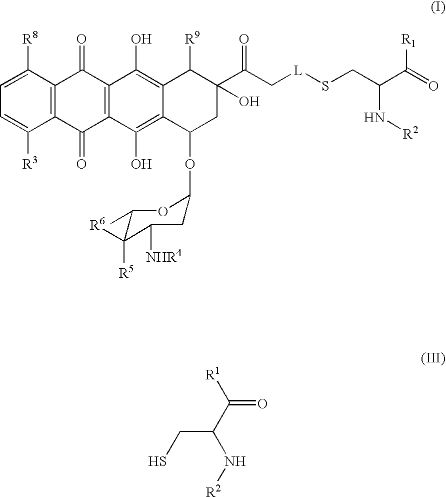
wherein L represents a linker arm of the formula R—X—Y—, wherein R is —O—C(═O)—, Y is the product of Z upon reaction with the thiol moiety of compound of formula (III) and X, R¹, R², R³, R⁴, R⁵, R⁶, R⁸, R⁹ and R¹⁰ have the same meaning as that defined above.

3. Method according to claim 1, comprising the step of
a) halogenating the compound of formula (II), resulting in compound of formula (IIa), 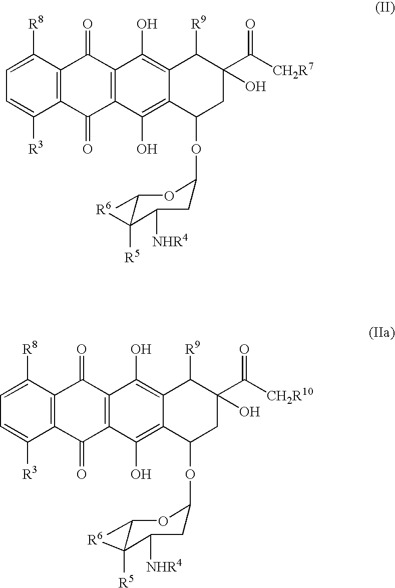
b) reacting the compound of formula (IIa) at its 14 position with the thiol moiety of a peptide of formula (III) to obtain compound of formula (I) 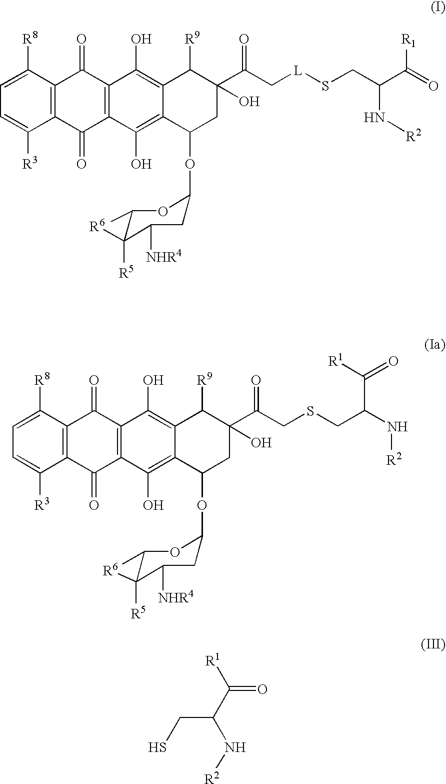
wherein R¹, R², R³, R⁴, R⁵, R⁶, R⁸, R⁹ and R¹⁰ have the same meaning as that defined above and -L- is absent as represented by formula (Ia).

4. Method according to claim 1, wherein R¹⁰ is Br.

5. Method according to claim 1, wherein the halogenation step is done simultaneously with a ketalization step of the 13-ketone of the compound of formula (II) in the presence of a suitable alcohol.

6. Method according to claim 5, wherein the ketalization step is performed in the presence of a suitable orthoester.

7. Method according to claim 2, wherein the functional group Z is selected from the group consisting of α,β-unsaturated carbonyl, carboxy, carbamoyl and imidyl radical.

8. Method according to claim 7, wherein the functional group Z is a maleimidyl radical.

9. Method according to claim 2, wherein said linker of formula (IV) is maleimidobutyric acid.

10. Method according to claim 1, wherein the compound of formula (II) is daunorubicin, carminomycin or idarubicin.

11. Method according to claim 10, wherein the compound of formula (II) is daunorubicin.

12. Method according to claim 1, wherein the peptide of formula (III) contains from 1 to 100 amino acids.

13. Method according to claim 12, wherein the peptide of formula (III) contains from 10 to 30 amino acids.

14. Method according to claim 1, wherein the compound of formula (I) is a compound of formula (Id) 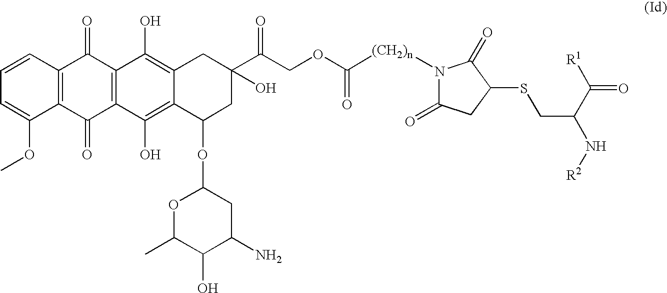
wherein R¹ and R² have the same meaning as that defined above and n is a number ranging from 2 to 10.

15. Method according to claim 14, wherein the compound of formula (Id) is a compound of formula (Ic) 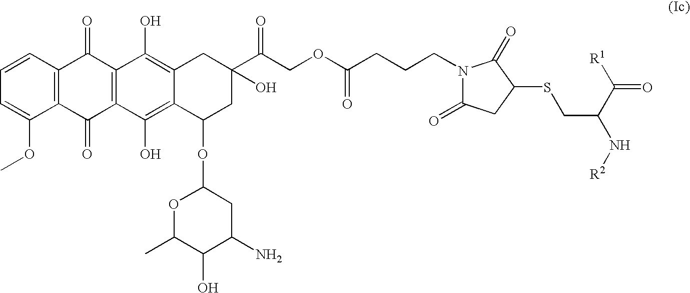
wherein R¹ and R² have the same meaning as that defined above.

16. An intermediate obtained by the method of claim 1.

17. A compound obtained by the method of claim 1.

18. A compound having the formula (Ia), 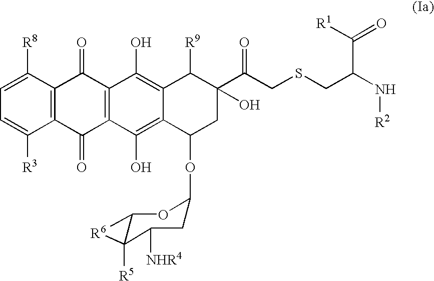
wherein R³ represents OCH₃, OH or H, R⁴ represents H or COCF₃, R⁵ represents OH, O-tetrahydropyranyl or H, R⁶ represents OH or H, R⁸ represents OH or H, R⁹ represents OH or H; R¹ represents OH, NH₂ or NH-peptide and R² represents H or —CO-peptide.

19. The compound according to claim 18, wherein R³ represents OCH₃, OH or H, R⁴ represents H, R⁵ represents OH, O-tetrahydropyranyl or H, R⁶ represents OH or H, R⁸ is H, R⁹ is H; R¹ represents OH, NH₂ or NH-peptide and R² represents H or —CO-peptide.

20. The compound according to claim 19, wherein R³ represents OCH₃, OH or H, R⁴ is H, R⁵ is OH, R⁶ is H, R⁸ is H, R⁹ is H; R¹ represents OH, NH₂ or NH-peptide and R² represents H or —CO-peptide.

21. The compound according to claim 20, having the formula (Ib), 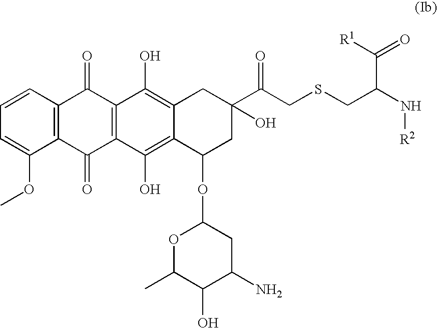
wherein R¹ and R² have the same meaning as that defined above.

22. The compound according to claim 18, wherein said compound contains from 1 to 100 amino acids.

23. The compound according to claim 22, wherein said compound contains from 10 to 30 amino acids.

24. A pharmaceutical composition comprising a pharmaceutical carrier and a therapeutically effective amount of a compound according to claim 18.

25. (canceled)

26. A method of treating a tumor which comprises administering a therapeutically effective amount of a compound according to claim 18 to a patient in need thereof.

27. A method of preparing an antitumor agent which comprises using the compound according to claim 16 as a precursor.

28. A method of treating cancer which comprises administering a therapeutically effective amount of the compound according to claim 17 to a patient in need thereof.
